(19) United States
(12) Patent Application Publication
(10) Pub. No.: US 2018/0028059 A1
(43) Pub. Date: Feb. 1, 2018

(54) AN APPARATUS FOR MULTI-MODE IMAGING OF EYE

(71) Applicant: FORUS HEALTH PRIVATE LIMITED, BANGALORE (IN)

(72) Inventors: SANTOSH KUMAR COTHURU, BANGALORE (IN); MAHABALESWARA RAM BHATT, BANGALORE (IN); SHYAM VASUDEVA RAO, BANGALORE (IN); POSTON TIMOTHY, BANGALORE (IN)

(73) Assignee: FORUS HEALTH PRIVATE LIMITED, BANGALORE (IN)

(21) Appl. No.: 15/550,338

(22) PCT Filed: May 20, 2015
(86) PCT No.: PCT/IB2015/053710

§ 371 Date: Aug. 10, 2017

(30) Foreign Application Priority Data
Mar. 24, 2015 (IN) 1508/CHE/2015

(51) Int. Cl. A61B 3/18; A61B 3/00; G01J 3/28; A61B 3/12; G01B 9/02; H04N 5/225; A61B 3/14; A61B 3/10

(52) U.S. Cl. CPC A61B 3/18; A61B 3/14; A61B 3/0025; A61B 3/102; A61B 3/12; G01B 9/02044; G01B 9/02091; H04N 5/2256; G01J 3/2823; G01B 2290/65; G01B 2290/30; G01B 2290/70; G01J 2003/2826

(57) ABSTRACT

The present disclosure relates to an apparatus for multi-mode imaging of an eye. The apparatus integrates at least two modes namely OCT, hyper-/multi-spectral imaging, and RGB/IR. The apparatus captures high resolution and larger dynamic range OCT and hyper-/multi-spectral images, in a high dynamic range multi-wavelength image data. The apparatus captures the images through an optical path sharing one or more optical elements and a detection unit. The captured images are processed by a computing device associated with the apparatus for either OCT or hyper-spectral imaging.

### TECHNICAL FIELD

The present subject matter is related, in general to ophthalmic apparatus and more particularly, but not exclusively to an apparatus integrated with multiple modalities for imaging eye.

### BACKGROUND

The visual capacity of human eye is dependent on health of various tissue layers of the eye such as cornea, lens and retina. Damage to nerve fibers and blood vessels of the retina can progressively modify the tissue structure and lead to poor vision or blindness. Therefore, there is a requirement for affordable, low-power screening devices, for early detection of various eye ailments. Early symptoms of many eye ailments can be visualized as abnormal changes in the retinal or corneal structure. At present, diagnosis requires use of a variety of image forming models and acquisition techniques (‘modalities’), each with its own physical apparatus and associated with embedded software. For example, the modalities may be a Frequency Domain-Optical Coherence Tomography (FD-OCT) imaging, a hyper/multi-spectral imaging and a Red Green Blue/infrared (RGB/IR) imaging.

At present, it is typical for each such modality to be handled by a separate apparatus. Each apparatus has its own requirements of filters, mirrors, light emitters, detectors, and so on, along optical path followed by optical signals. If multiple modalities are merged in a single apparatus, the elements needed for each modality can block the optical path for another. Moreover, each imaging technology produces an image with a different field of view, resolution, and size for the features of interest. Apart from this difficulty, the cost of the multiple items of specialized apparatus makes the diagnostic process less affordable. Moreover, the system is often bulky and not portable.

In particular, the existing technique provides separate equipment for OCT imaging, hyper-spectral imaging, multi-spectral and RGB/IR imaging. However, a single imaging device is often insufficient to enable a preliminary diagnosis or confirmation of symptoms related to a particular ophthalmic or systemic ailment. As a result, multiple devices are required. This leads to a great amount of waiting for a patient between the successive eye tests and inefficiency in clinical scheduling as the patient has to wait at each equipment for observation. Further, these factors make it problematic to deploy such non-portable apparatus in public health screening camps, both logistically and in the financial risk of damage.

### SUMMARY

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

Accordingly, the present disclosure relates to an apparatus for multi-mode imaging of an eye. The apparatus comprises a first source component, a second source component, a coupler, a polarizing mirror, a first digital micro mirror device, a second digital micro mirror device, a combining component and a detection unit. The first source component generates an illumination light. The second source component generates a white light. The coupler is configured to receive the illumination light from the first source component and split the illumination light into a first light and a second light. The coupler projects the first light onto a reflecting mirror and the second light onto the eye. The coupler receives the first light reflected from the reflecting mirror and the second light reflected from the eye and combines the reflected first light and the reflected second light to form an interference pattern. The polarizing mirror receives the white light from the second source component and projects the white light onto the eye through a focusing lens. The first digital micro mirror device, comprising one or more first mirrors, receives the white light reflected from the eye through the polarizing mirror and scatters the reflected white light. The combining component performs at least one of receiving the interference pattern from the coupler when a first mode is active or receiving the reflected white light from the first digital micro mirror device when a second mode is active. The detection unit comprises a diffraction grating, a second digital micro mirror device, a photo detector and a digitizer. The diffraction grating receives at least one of the interference pattern or the white light from the combining component and diffracts at least one of the interference pattern and the white light into one or more spectral components. The second digital micro mirror device comprising one or more second mirrors receives each of the one or more spectral components from the diffraction grating and scatters each of the one or more spectral components. The photo detector is configured to receive each of the scattered one or more spectral components and convert each of the scattered one or more spectral components into an analog signal. The digitizer converts the analog signal into a digital signal, wherein the digital signal is processed by a computing device associated with the apparatus used for multi-mode imaging of the eye.

Further, the present disclosure relates to a method for multi-mode imaging of an eye using an apparatus. The method comprises receiving, by a combining component, at least one of an interference pattern from a coupler when a first mode is active or a reflected white light scattered from a first digital micro mirror device, comprising one or more first mirrors, when a second mode is active. The method further comprises projecting, by the combining component, at least one of the interference pattern or the white light onto a diffraction grating for diffracting into one or more spectral components. Upon receiving at least one of the interference pattern or the white light, the diffraction grating diffracts each of the one or more spectral components onto a second digital micro mirror device wherein the second digital micro mirror device comprises one or more second mirrors. The second digital micro mirror device scatters each of the one or more spectral components. The method further comprises projecting, by the second digital micro mirror device, each of the one or more scattered spectral components onto a photo detector. The photo detector converts each of the one or more scattered spectral components into an analog signal. The digitizer converts the analog signal into a digital signal for multi-mode imaging of the eye.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 shows a block diagram illustrating an apparatus for performing FD-OCT imaging of the eye in accordance with some embodiments of the present disclosure;

FIG. 2 shows a block diagram illustrating an apparatus for performing hyper spectral/multispectral/RGB/IR imaging of the eye in accordance with some embodiments of the present disclosure;

FIG. 3 shows a block diagram illustrating an apparatus for multi-mode imaging of the eye in accordance with some embodiments of the present disclosure;

FIG. 4 shows a block diagram illustrating an apparatus for multi-mode imaging of the eye using a plane mirror in accordance with some embodiments of the present disclosure; and

FIG. 5 illustrates a flowchart showing method for performing multi-mode imaging of eye in accordance with some embodiments of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

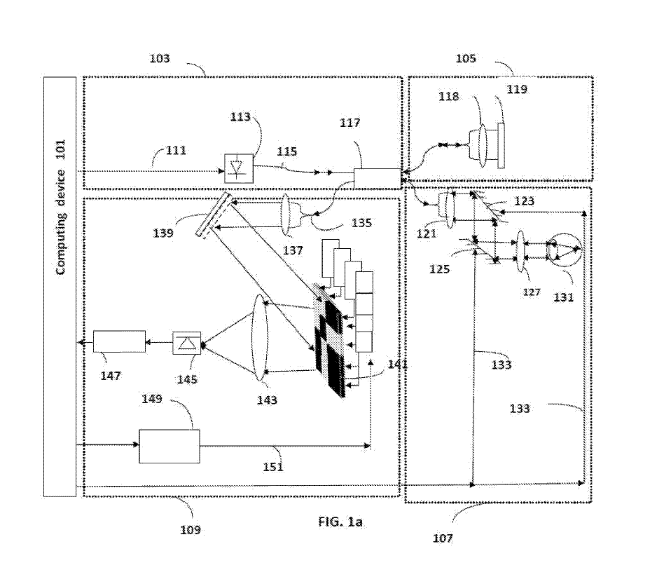

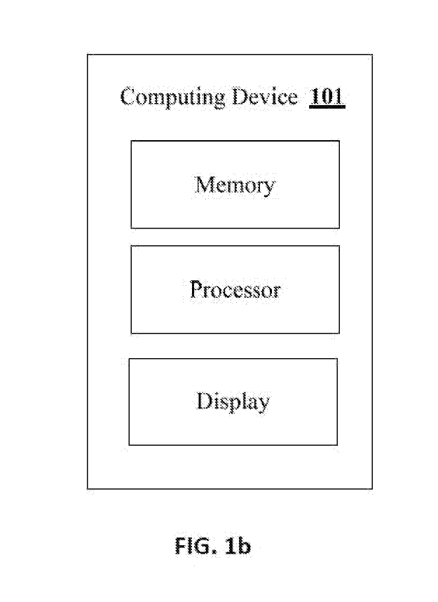

FIG. 1a shows a block diagram illustrating an apparatus for performing FD-OCT imaging of the eye in accordance with some embodiments of the present disclosure.

In FD-OCT imaging mode, the apparatus captures a cross-section or 3-dimensional image of a shallow region of the eye. For eye imaging, this image provides information of detailed structure in multiple layers of the cornea, lens or retina, with great diagnostic value in detecting lesions or other abnormalities in eye. The FD-OCT imaging mode is basically used for telemedicine based screening and management of Age-related Macular Degeneration (AMD) and other retinal diseases. In an embodiment, the apparatus with FD-OCT imaging mode assesses the microscopic structure of intra-retinal layers and choroidal vessel system.

As shown in FIG. 1a, the apparatus comprises a first source component 103, a reference unit 105, a sample unit 107 and a detection unit 109. The apparatus is associated with a computing device 101. The computing device 101 comprises a processor, a memory and a display as shown in FIG. 1b. The first source component 103 comprises a diode 113 and a coupler 117. The reference unit 105 comprises a first collimating lens 118 and a reflecting mirror 119. The sample unit 107 comprises a second collimating lens 121, a first galvano-mirror 123, a second galvano-mirror 125, a first focussing lens 127 and a target 131. The detection unit 107 comprises a third collimating lens 137, a diffraction grating 139, a second digital micro mirror device 141, a second focusing lens 143, a photo detector 145, a digitizer 147 and a second digital micro mirror controller 149.

In an embodiment, the computing device 101 provides an illumination generation signal 111 to the diode 113 for generating an illumination light. The diode 113 generates an illumination light. As an example, wavelength of the generated illumination light is 840 nm. The wavelength of the generated illumination light may vary based on band range of the illumination light. The illumination light is guided onto the coupler 117 through an optical fibre 115. Upon receiving the illumination light, the coupler 117 splits the illumination light into a first light and a second light. In an embodiment, wavelength of the first light and the second light are equal. The coupler 117 projects the first light onto the reflecting mirror 119 through the first collimating lens 118 and projects the second light onto the second collimating lens 121 for collimating the second light. The reflecting mirror 119 reflects the first light back to the coupler 117 through the collimating lens 118. The second collimating lens 121 guides the second light onto the first galvano-mirror 123. As an example, a polygon mirror may be used in the apparatus instead of galvano-mirror. The first galvano-mirror 123 deflects the second light and the deflected second light is directed towards the second galvano-mirror 125. In an embodiment, the first galvano mirror 123 and the second galvano mirror 125 can be combined and a 2D Micro Electro Mechanical Systems (MEMS) mirror may be used which functions as an x-y scanner. The second galvano-mirror 125 deflects the second light onto a first focussing lens 127. The first galvano-mirror 123 and the second galvano-mirror 125 are controlled by the computing device 101 though the controlling signals 133. In an embodiment, the movement of the first focussing lens 127 is controlled by the computing device 101. In another embodiment, the movement of the first focussing lens 127 can be set manually. The first focussing lens 127 projects the second light onto the target 131. As an example, the target may be cornea of the eye or retina of the eye. The second light reflected from the eye is projected towards the coupler 117 through the first focusing lens 127, the second galvano-mirror 125, the first galvano-mirror 123 and the second collimating lens 121. The coupler 117 combines the reflected first light and the reflected second light to form an interference pattern. The coupler 117 guides the interference pattern towards the detection unit 109. The coupler 117 projects the interference pattern onto the third collimator lens 137 through a unidirectional fibre 135. The third collimating lens 137 projects the interference pattern onto the diffraction grating 139. The diffraction grating 139 is configured to disperse and diffract the light into components which travel in distinct directions. The components are dependent on their wavelength and spacing between lines on the diffraction grating 139. The diffraction grating 139 may be either reflective or refractive type. The diffraction grating 139 diffracts the interference pattern into one or more spectral components. The one or more spectral components are received by the second digital micro mirror device 141. The second digital micro mirror device 141 comprises of one or more second mirrors. The activation or deactivation or orientation of each of the one or more second mirrors is controlled by the second digital micro mirror controller 149 connected to the computing device 101. The second digital micro mirror controller 149 generates a mirror controlling signal 151 at one or more time intervals based on which the one or more second mirrors are activated. In an embodiment, the one or more second mirrors may be placed in a way that the mirrors face towards the photo detector 145 or face away from the photo detector 145.

The one or more second mirrors in the second digital micro mirror device 141 scatter each of the one or more spectral components. The second focussing lens 143 is placed between the second digital micro mirror device 141 and the photo detector 145. The second digital micro mirror device 141 projects each of the scattered one or more spectral components through the second focussing lens 143 on the photo detector 145. Upon receiving the scattered spectral components, the photo detector 145 converts the optical signal i.e the scattered spectral components into an analog signal. The photo detector 145 is connected to a digitizer 147. The digitizer 147 receives the analog signal from the photo detector 145 and digitizes the analog signal. The digital signal corresponds to number of pixels in the image of the eye. The digital signal is used to assess the microscopic structure of intra-retinal layers and inter-corneal layers of the eye. In an embodiment, the digital signal is used to reconstruct the OCT image by the computing device 101 and the reconstructed image is displayed on the display of the computing device 101.

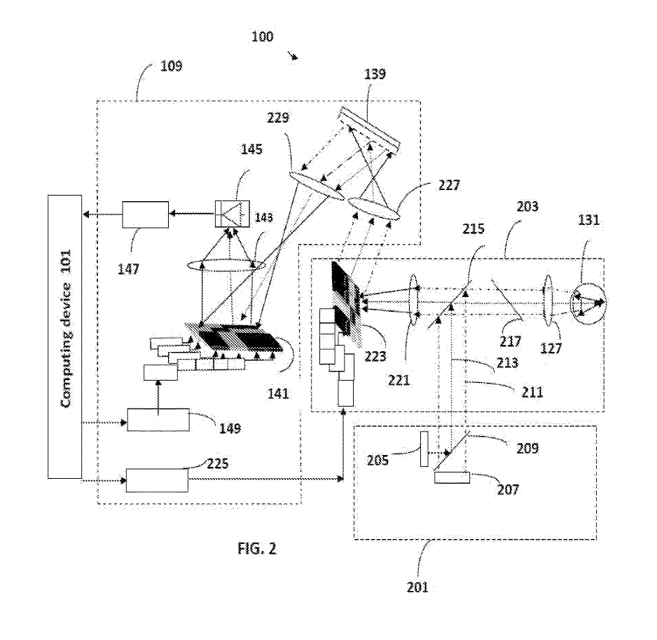

FIG. 2 shows a block diagram illustrating the apparatus for performing hyper spectral/multispectral/RGB/IR imaging of the eye in accordance with some embodiments of the present disclosure.

In hyper spectral/multispectral/RGB/IR imaging mode, the apparatus identifies retinal blood vessels, the optic disc, oxygen saturation levels, and pathological indicators of various eye ailments, such as macular pigments, drusen, etc. Also, RGB imaging helps to locate the optic disc, macula, posterior pole, retinal blood vessels, drusen, pigmentation, etc.

The apparatus comprises a second source component 201, a sample unit 203 and a detection unit 109. The apparatus is connected to the computing device 101. The second source component 201 comprises an IR light source 205, a white light source 207 and a hot mirror 209. The IR light source 205 produces an IR light 213 and the white light source 207 produces a white light 211. The white light 211 and the IR light 213 are mixed by the hot mirror 209. The sample unit 203 comprises a first digital micro mirror device 223, a fourth collimating lens 221, a polarizing mirror 215, an optical element 217, the first focusing lens 127 and the target 131. The detection unit 109 comprises the diffraction grating 139, lens 227, lens 229, the second digital micro mirror device 141, the focusing lens 143, the photo detector 145, the digitizer 147, the first digital micro mirror controller 225 and the second digital micro mirror controller 149.

In an embodiment, the hot mirror 209 reflects the IR light 213 and directs the white light 211 to the polarizing mirror 215. The polarizing mirror 215 projects the white light 211 onto the eye 131 through the optical element 217 and the first focusing lens 127. The optical element 217 is placed in between the polarizing mirror 215 and the first focusing lens 127. The eye 131 reflects the white light 211 and the reflected white light 211 is projected on the first digital micro mirror device 223 through the collimating lens 221. The first digital micro mirror device 223 comprises of one or more first mirrors. The activation/deactivation of the one or more first mirrors is controlled by the first digital micro mirror controller 225 connected to the computing device 101. The first digital micro mirror device 223 reflects the white light and the reflected white light is projected onto the diffraction grating 139 through the relay lens 227. The diffraction grating 139 scatters the white light into one or more spectral components. The one or more spectral components correspond to components of a hyper spectral image. Each of the one or more spectral components is projected onto a second digital micro mirror device 141 through relay lens 229. The second digital micro mirror device 141 comprises of one or more second mirrors. The activation/deactivation/orientation of each of the one or more second mirrors are controlled by the second digital micro mirror controller 149 connected to the computing device 101. The one or more second mirrors of the second digital micro mirror device 141 are used as a reflection based pixel wise light projector which scatters the spectral components and the scattered spectral components are projected onto the photo detector 145 through the second focusing lens 143. The photo detector 145 converts the scattered spectral components/optical signals into an analog signal. The analog signal is passed to the digitizer 147. The digitizer 147 converts the analog signal into a digital signal. The digital signal is processed by the computing device 101 for hyper spectral/multispectral/RGB/IR imaging of the eye. The digital signal is used by the computing device 101 for reconstructing the hyper spectral/multispectral/RGB/IR image. The reconstructed hyper spectral/multispectral/RGB/IR image is displayed on display of the computing device 101 for analyzing.

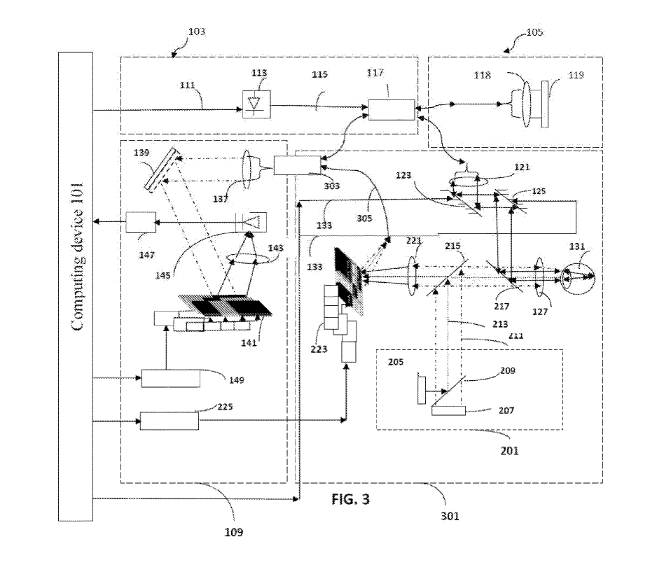

FIG. 3 shows a block diagram illustrating an apparatus for multi-mode imaging of the eye in accordance with some embodiments of the present disclosure.

In an embodiment, the apparatus is configured for multi-mode imaging of the eye. The multiple modes are RGB color retina or cornea image capturing mode, frequency domain (FD) optical coherence tomography (OCT) mode and 3-dimensional hyper spectral/3-dimensional multi-spectral mode. A user may select any of the above modes according to which the apparatus is implemented.

In an embodiment, the OCT mode is a first mode and the RGB color retina or cornea image capturing mode and 3-dimensional hyper spectral/3-dimensional multi-spectral mode is a second mode.

In an embodiment, the apparatus as shown in FIG. 3 is configured to perform multi-mode imaging of the eye without obstructing optical paths of each mode.

The apparatus comprises the first source component 103, the second source component 201, the reference unit 105, the sample unit 301, a combining component 303 and the detection unit 109. The apparatus is connected to the computing device 101. The first source component 103 comprises the diode 113 and the coupler 117. The second source component 201 comprises the IR light source 205, the white light source 207 and the hot mirror 209. The reference unit 105 comprises the first collimating lens 118 and the reflecting mirror 119. The sample unit 301 comprises the first galvano-mirror 123, the second galvano-mirror 125, the second collimating lens 121, the first optical element 217, the first focusing lens 127, the polarizing mirror 215, a fourth collimating lens 221 and the first digital micro mirror device 223. The detection unit 109 comprises the diffraction grating 139, the third collimating lens 137, the second digital micro mirror device 141, the second focusing lens 143, the photo detector 145, the digitizer 147, the first digital micro mirror controller 225 and the second digital micro mirror controller 149.

The first source component 103 is configured to generate the illumination light and the second source component 201 is configured to generate the white light. In an embodiment, when the first mode is activated, the apparatus functions as illustrated in FIG. 1.

The computing device 101 provides an illumination generation signal 111 to the diode 113 for generating the illumination light. The diode 113 generates the illumination light. As an example, wavelength of the generated illumination light is 840 nm. As an example, the wavelength of the generated illumination light may vary based on band range of the illumination light. The illumination light is guided onto the coupler 117 through an optical fibre 115. Upon receiving the illumination light, the coupler 117 splits the illumination light into a first light and a second light. The wavelength of the first light and the second light are equal. The coupler 117 projects the first light onto the reflecting mirror 119 through the first collimating lens 118 and projects the second light onto the second collimating lens 121 and then to the first galvano-mirror 123. The second light deflected from the first galvano-mirror 123 is projected onto the second galvano-mirror 125. In an embodiment, the first galvano mirror 123 and the second galvano mirror 125 can be combined and a 2D MEMS mirror may be used which functions as an x-y scanner. The second light deflected from the second galvano-mirror 125 is projected onto the first optical element 217. The first galvano-mirror 123 and the second galvano-mirror 125 are controlled by the computing device 101 through a controlling signal 133. The first optical element 217 is used to block the optical path for the second mode i.e when the first mode is active the first optical element 217 blocks the optical path from the second source component 201. The first optical element 217 projects the second light onto the eye 131 through the first focussing lens 127. The reflected first light from the eye 131 is received by the coupler 117 through the first focusing lens 127, the first optical element 217, the second galvano-mirror 125 and the first galvano-mirror 123.

The reflecting mirror 119 reflects the first light onto the coupler 117. The coupler 117 receives the reflected first light and the reflected second light. The coupler 117 combines the reflected first light and the reflected second light to form the interference pattern. The coupler projects interference pattern onto the combining component 303.

In an embodiment, when the second mode is activated, the apparatus functions as illustrated in FIG. 2.

The second source component 201 comprises the white light source 207 and the IR light source 205. The white light source 207 generates the white light 211 and the IR light source 205 generates the IR light 213. The white light 211 and the IR light 213 are mixed by the hot mirror 209. The hot mirror 209 reflects the IR light 213 and directs the white light 211 onto the polarizing mirror 215. The polarizing mirror 215 directs the white light 211 onto the eye 131 through the first focusing lens 127. The polarizing mirror 215 receives the reflected white light from the eye 131 and projects onto the first digital micro mirror device 223 through the fourth collimator lens 221. The first digital micro mirror device 223 comprises of one or more first mirrors. The activation/deactivation or orientation of the one or more first mirrors are controlled by the first digital micro mirror controller 225. The first digital micro mirror controller 225 is connected to the computing device 101. The computing device 101 provides a signal to the first digital micro mirror controller 225 based on which the first digital micro mirror controller 225 controls the activation/deactivation of the one or more first mirrors. The first digital micro mirror device 223 scatters the reflected white light and provides the reflected white light to the combining component 303 through the optical fibre 305.

The combining component 303 projects the interference pattern onto the diffraction grating 139 through the third collimator lens 137 when the first mode is active and projects the reflected white light onto the diffraction grating 139 through the third collimator lens 137 when the second mode is active. The diffraction grating 139 diffracts the interference pattern into one or more spectral components during the first mode. The diffraction grating 139 diffracts the reflected white light into one or more spectral components during the second mode. The second digital micro mirror device 141 receives each of the one or more spectral components from the diffraction grating 139. The second digital micro mirror device 141 comprises of one or more second mirrors. The activation/deactivation or orientation of the one or more second mirrors is controlled by the second digital micro mirror controller 149. The second digital micro mirror controller 149 is connected to the computing device 101. The spectral components reflected from each of the one or more second mirror corresponds to each pixel of the image. In an embodiment, the second digital micro mirror device 141 is used as a reflection based pixel wise light projector which scatters the spectral components. The second digital micro mirror device 141 scatters each of the one or more spectral components and the scattered each of the one or more spectral components is provided to the photo detector 145 through the second focusing lens 143. The photo detector 145 converts the optical signal into analog signal. The digitizer 147 receives the analog signal and converts into digital signal. The digital signal is processed by the computing device 101 for multi-mode imaging of the eye.

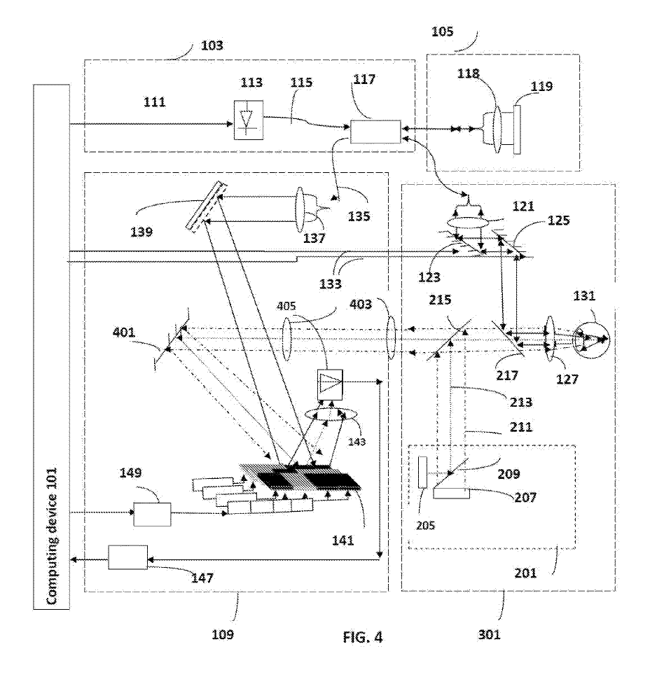

FIG. 4 shows a block diagram illustrating an apparatus for multi-mode imaging of the eye using a plane mirror 401 in accordance with some embodiments of the present disclosure.

In one implementation, the apparatus shown in FIG. 4 is an alternative embodiment of the apparatus illustrated in FIG. 3. The apparatus illustrated in FIG. 3 uses two digital micro mirror devices namely the first digital micro mirror device 223 and the second digital micro mirror device 141. The first digital micro mirror device 223 is configured to receive reflected white light from the eye and to provide the reflected white light to the combining component 303. The second digital micro mirror device 141 is configured to receive each of the one or more spectral components and scatter each of the one or more spectral components. The apparatus as shown in FIG. 4 uses a plane mirror when the second mode is active for receiving the reflected white light from the eye. The first digital micro mirror device 223 is removed from the apparatus illustrated in FIG. 4. The plane mirror 401 receives the reflected white light from the eye through the relay compound lens 403 and 405 and projects the reflected white light onto the second digital micro mirror device 141. The apparatus as shown in FIG. 4 does not use the combining component as illustrated in FIG. 3 as the plane mirror 401 receives the reflected white light and projects directly onto the second digital micro mirror device 141. The functions of the apparatus as shown in FIG. 4 when the first mode is active are as illustrated in FIG. 1. The functions of the apparatus as shown in FIG. 4 when the second mode is active are as illustrated in FIG. 2.

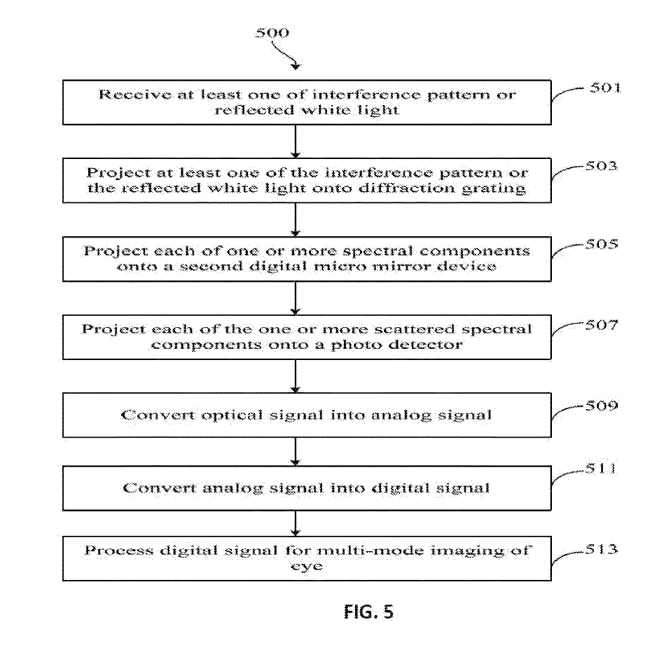

FIG. 5 illustrates a flowchart showing method for performing multi-mode imaging of eye in accordance with some embodiments of the present disclosure.

As illustrated in FIG. 5, the method 500 comprises one or more blocks for performing multi-mode imaging of eye using an apparatus as illustrated in FIG. 3. The method may be described in the general context of computer executable instructions. Generally, computer executable instructions can include routines, programs, objects, components, data structures, procedures, modules, and functions, which perform particular functions or implement particular abstract data types.

The order in which the method 500 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

When a user selects the first mode, the apparatus is implemented to perform the OCT imaging modality. When the user selects the second mode, the apparatus is implemented to perform RGB color retina or cornea image capturing mode and 3-dimensional hyper spectral/3-dimensional multi-spectral mode is a second mode. The apparatus comprises the first source component 103, the second source component 201, the reference unit 105, the sample unit 301, a combining component 301 and the detection unit 109. The apparatus is connected to the computing device 101. The first source component 103 comprises the diode 113 and the coupler 117. The second source component 201 comprises the IR light source 205, the white light source 207 and the hot mirror 209. The reference unit 105 comprises the first collimating lens 118 and the reflecting mirror 119. The sample unit 105 comprises the first galvano-mirror 123, the second galvano-mirror 123, the first optical element 217, the first focusing lens 127, the polarizing mirror 215 and the first digital micro mirror device 223. The detection unit 109 comprises the diffraction grating 139, the third collimating lens 137, the second digital micro mirror device 141, the second focusing lens 143, the second digital micro mirror device 141, the photo detector 145, the digitizer 147, the first digital micro mirror controller 225 and the second digital micro mirror controller 149.

The first source component 103 is configured to generate the illumination light and the second source component is configured to generate the white light.

The computing device 101 provides a signal 111 to the diode 113 for generating the illumination light. The diode 113 generates the illumination light. The illumination light is guided onto the coupler 117 through the optical fibre 115. Upon receiving the illumination light, the coupler 117 splits the illumination light into a first light and a second light. The wavelength of the first light and the second light are equal. The coupler 117 projects the first light onto the reflecting mirror 119 through the first collimating lens 118 and projects the second light onto the first galvano-mirror 123. The second light deflected from the first galvano-mirror 123 is projected onto the second galvano-mirror 125. The second light deflected from the second galvano mirror 125 is projected onto the first optical element 217. The first optical element 217 projects the second light onto the eye 131 through the first focussing lens 127. The reflected second light from the eye is received by the coupler 117 through the first focusing lens 127, the first optical element 217, the second galvano-mirror 125 and the first galvano-mirror 123.

The reflecting mirror 119 reflects the first light onto the coupler 117. The coupler 117 receives the reflected first light and the reflected second light. The coupler 117 combines the reflected first light and the reflected second light to form the interference pattern. The coupler 117 projects the interference pattern to the combining component 303.

The second source component 201 comprises the white light source 207 and the IR light source 205. The white light source 207 generates the white light 211 and the IR light source 205 generates the IR light 213. The white light 211 and the IR light 213 are mixed by the hot mirror 209. The hot mirror 209 reflects the IR light 213 and directs the white light 211 onto the polarizing mirror 215. The polarizing mirror 215 directs the white light 211 onto the eye 131 through the first focusing lens 127. The polarizing mirror 215 receives the reflected white light from the eye 131 and projects onto the first digital micro mirror device 223 through the fourth collimator lens 221. The first digital micro mirror device 223 scatters the reflected white light and provides the reflected white light to the combining component 303 through the optical fibre 305.

At block 501, the combining component 303 receives at least one of the interference patterns from the coupler 117 when the first mode is active and the reflected white light from the first digital micro mirror device 223 when the second mode is active.

At block 503, the combining component 303 projects the interference pattern on to the diffraction grating 139 through the third collimator lens 137 when the first mode is active and projects the reflected white light onto the diffraction grating 139 through the third collimator lens 137 when the second mode is active. The diffraction grating 139 diffracts the interference pattern or the reflected white light into one or more spectral components.

At block 505, the diffraction grating 139 projects the one or more spectral components to the second digital micro mirror device 141. The second digital micro mirror device 141 scatters each of the one or more spectral components.

At block 507, the second digital micro mirror device 141 projects each of the scattered one or more spectral components to the photo detector 145 through the second focusing lens 143.

At block 509, the photo detector 145 converts the optical signal into analog signal.

At block 511, the digitiser 147 receives the analog signal and converts into digital signal.

At block 513, the computing unit 101 receives the digital signal and processes the digital signal for multi-mode imaging of the eye.

### Advantages of the Embodiment of the Present Disclosure are Illustrated Herein

In an embodiment, the present disclosure provides a single apparatus for FD-OCT imaging, 3-Dmulti spectral imaging, RGB/IR imaging and 3-D hyper spectral imaging.

In an embodiment, the optical elements used for each image capturing mode does not reduce the quality of the image captured by another mode.

In an embodiment, the present disclosure provides a single cost-effective, affordable and portable apparatus for performing multiple modalities.

In an embodiment, there is no need for a patient to be moved from one apparatus to another apparatus while being diagnosed.

In an embodiment, the present disclosure provides a method for capturing the images and displaying on the display screen instead of ophthalmoscope based detection by clinician's eye which cannot perceive ultrasound or infrared radiations.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Referral Numerals:

| Reference Number | Description |
|---|---|
| 101 | Computing device |
| 103 | First source component |
| 105 | Reference unit |
| 107 | Sample unit |
| 109 | Detection unit |
| 111 | Illumination Generation signal |
| 113 | diode |
| 115 | Optical fibre |
| 117 | coupler |
| 118 | First collimator lens |
| 119 | Reflecting mirror |
| 121 | Second collimator lens |
| 123 | First galvano-mirror |
| 125 | Second galvano-mirror |
| 127 | First focusing lens |
| 131 | Target/eye |
| 133 | Controlling signal |
| 135 | Unidirectional fibre |
| 137 | Third collimator lens |
| 139 | Diffraction grating |
| 141 | Second digital micro mirror device |
| 143 | Second focusing lens |
| 145 | Photo detector |
| 147 | digitizer |
| 149 | Second digital micro mirror controller |
| 151 | Mirror controlling signal |
| 201 | Second source component |
| 203 | Sample unit |
| 205 | IR light source |
| 207 | White light source |
| 209 | Hot mirror |
| 211 | White light |
| 213 | IR light |
| 215 | Polarizing mirror |
| 217 | First optical element |
| 221 | Fourth collimator lens |
| 223 | Second digital micro mirror device |
| 225 | Second digital micro mirror controller |
| 301 | Sample unit |
| 303 | Combining component |
| 305 | Optical fibre |
| 401 | Plane mirror |
| 403, 405 | Relay Compound lens |

1. An apparatus for multi-mode imaging of an eye, the apparatus comprising:
a first source component configured to generate an illumination light;
a second source component configured to generate a white light;
a coupler configured to:
receive the illumination light from the first source component;
split the illumination light into a first light and a second light, wherein the first light is projected onto a reflecting mirror and the second light is projected onto the eye;
receive the first light reflected from the reflecting mirror and second light reflected from the eye; and
combine the reflected first light and the reflected second light to form an interference pattern;
a polarizing mirror configured to receive the white light from the second source component and project the white light onto the eye through a focusing lens;
a first digital micro mirror device, comprising one or more first mirrors, configured to receive the white light reflected from the eye through the polarizing mirror and scatter the reflected white light;
a combining component configured to perform at least one of:
receiving the interference pattern from the coupler when a first mode is active; or
receiving the reflected white light from the first digital micro mirror device when a second mode is active; and
a detection unit comprising:
a diffraction grating configured to:
receive at least one of the interference pattern or the white light from the combining component and diffract at least one of the interference pattern and the white light into one or more spectral components;
a second digital micro mirror device, comprising one or more second mirrors, configured to receive each of the one or more spectral components from the diffraction grating and scatter each of the one or more spectral components;
a photo detector, configured to receive each of the scattered one or more spectral components and convert each of the scattered one or more spectral components into an analog signal; and
a digitizer for converting the analog signal into a digital signal, wherein the digital signal is used for multi-mode imaging of the eye.

2. The apparatus as claimed in claim 1, wherein the first mode is three dimensional image capturing mode and the second mode is three dimensional multi-spectral image capturing mode.

3. The apparatus as claimed in claim 1, wherein wavelength of the generated illumination light is 840 nm.

4. The apparatus as claimed in claim 1, wherein the first source component generates the illumination light through a diode.

5. The apparatus as claimed in claim 1, wherein the first light is projected onto the reflecting mirror through a first collimator lens.

6. The apparatus as claimed in claim 1, wherein the second light is projected onto the eye through a first galvano-mirror, a second galvano-mirror, a second collimator lens and a short-pass mirror.

7. The apparatus as claimed in claim 1, wherein the first light and the second light comprise equal amount of the illumination light.

8. The apparatus as claimed in claim 1, wherein the second source component comprises a while light source for generating the white light.

9. The apparatus as claimed in claim 1, wherein the polarizing mirror receives the reflected white light from the eye through the short pass mirror.

10. The apparatus as claimed in claim 9, wherein the short-pass mirror allows the reflected white light to pass through completely and project on to the polarizing mirror.

11. The apparatus as claimed in claim 1, wherein a third collimator lens is placed between the polarizing mirror and the first digital micro mirror device for guiding the reflected white light from the polarizing mirror onto the first digital micro mirror device.

12. The apparatus as claimed in claim 1, wherein orientation of the one or more first mirrors in the first digital micro mirror device is controlled by a first digital micro mirror controller and a computing device associated with the unified apparatus.

13. The apparatus as claimed in claim 12, wherein the computing device is further configured to process the digital signal and reconstruct image of the eye to obtain at least one of a three dimensional image of the eye and three dimensional multi-spectral image of the eye.

14. The apparatus as claimed in claim 1, wherein orientation of the one or more second mirrors in the second digital micro mirror device is controlled by a second digital micro mirror controller and the computing device.

15. The apparatus as claimed in claim 14, wherein the orientation of the one or more second mirrors in the second digital micro mirror device is at least one of towards or away from the photo detector.

16. The apparatus as claimed in claim 1, wherein a plane mirror is configured for receiving the reflected white light from the eye and for projecting the reflecting white light onto the second digital micro mirror device.

17. A method for multi-mode imaging of an eye using an apparatus, the method comprising:
receiving, by a combining component, at least one of an interference pattern from a coupler when a first mode is active or a reflected white light scattered from a first digital micro mirror device, comprising one or more first mirrors, when a second mode is active;
projecting, by the combining component, at least one of the interference pattern or the white light onto a diffraction grating for diffracting into one or more spectral components;
projecting, by the diffraction grating, each of the one or more spectral components onto a second digital micro mirror device, comprising one or more second mirrors, for scattering each of the one or more spectral components;
projecting, by the second digital micro mirror device, each of the one or more scattered spectral components onto a photo detector for converting each of the one or more scattered spectral components into an analog signal; and
converting, by a digitizer, the analog signal into a digital signal for multi-mode imaging of the eye.

18. The method as claimed in claim 17, wherein the first mode is three dimensional image capturing mode and the second mode is three dimensional multi-spectral image capturing mode.

19. The method as claimed in claim 17, wherein the interference pattern is received by the combining component by performing one or more operations comprising:
receiving, by the coupler, an illumination light from a first source component;
splitting, by the coupler, the illumination light into a first light and a second light, wherein the first light is projected onto a reflecting mirror and the second light is projected onto the eye;
receiving, by the coupler, the first light reflected from the reflecting mirror and the second light reflected from the eye; and
combining, by the coupler, the reflected first light and the reflected second light to form the interference pattern.

20. The method as claimed in claim 17, wherein the reflected white light is received by the combining component by performing one or more operations comprising:
receiving, by a polarizing mirror, a white light from a second source component;
projecting, by the polarizing mirror, the white light onto the eye through a focusing lens; and
receiving, by the first digital micro mirror device, the white light reflected from the eye through the polarizing mirror and scattering the reflected white light onto the combining component.
